(19) United States
(12) Patent Application Publication
(10) Pub. No.: US 2016/0199043 A1
(43) Pub. Date: Jul. 14, 2016

(54) Collection Apparatus with Stacked Rings

(71) Applicant: Michael Koretsky, Hightstown, NJ (US)

(72) Inventor: Michael Koretsky, Hightstown, NJ (US)

(21) Appl. No.: 14/993,947

(22) Filed: Jan. 12, 2016

Related U.S. Application Data
(60) Provisional application No. 62/102,447, filed on Jan. 12, 2015.

(51) Int. Cl. A61B 10/00; A61H 19/00

(52) U.S. Cl. CPC A61B 10/0058; A61H 19/32

(57) ABSTRACT

A sperm collecting apparatus includes a plurality of rings wherein each ring includes an inner sleeve comprising a gel-like material. The inner sleeve defines an internal insertion channel portion that is sized for closely receiving a human penis. Each ring can be placed in a concentric stacked relationship with other similar rings so that the insertion channel portions of the rings form an elongated insertion channel. A connecting case holds the rings in the concentric stacked relationship. The length of the insertion channel can be adjusted by varying the number of rings in the concentric stacked relationship. Each of the rings is removable from and replaceable within the connecting case.

### RELATED APPLICATION

This application claims the benefit of U.S. Provisional Application No. 62/102,447, filed Jan. 12, 2015, entitled “Collection Apparatus with Stacked Rings,” which is incorporated herein in its entirety by this reference.

### BACKGROUND

The present invention relates to a sperm collecting apparatus. In particular, the present invention relates to such an apparatus that includes a plurality of stacked concentric rings.

Sperm collection devices have been previously known. Examples of such devices can be seen in U.S. Pat. Nos. 5,807,360, 7,753,895 and 7,993,315. Typically, such devices have a gel-like inner sleeve disposed within a housing and defining an insertion opening and channel for receiving a user's penis.

It is an object of the present invention to provide an improved sperm collection apparatus that can be easily modified by a user to vary the configuration and length of the insertion channel.

Additional objectives and advantages of the invention will be set forth in the description that follows, and in part will be apparent from the description, or may be learned by practice of the invention. The objects and advantages of the invention may be realized and obtained by means of the instrumentalities and combinations pointed out in this specification, including the appended claims.

### SUMMARY

To achieve the foregoing objectives, and in accordance with the purposes of the invention as embodied and broadly described in this document, there is provided a sperm collecting apparatus that includes a plurality of rings wherein each ring includes an inner sleeve comprising a gel-like material and wherein the inner sleeve defines an internal insertion channel portion that is sized for closely receiving a human penis. Each ring is configured so that it can be placed in a concentric stacked relationship with each of the other rings so that the insertion channel portions of the rings form an elongated insertion channel. A connecting case is configured to hold the plurality of rings in the concentric stacked relationship. In this configuration the length of the insertion channel can be adjusted by varying the number of rings in the concentric stacked relationship.

According to one embodiment, the connecting case extends around the circumference of each of the plurality of rings by 180 degrees or more. The connecting case can be made of a hard plastic that is sufficiently flexible to allow the connecting case to clip onto and hold each of the plurality of rings. The inner sleeve of each ring is held within a ring top portion and a ring bottom portion. Each ring includes a peripheral groove configured to mate with a rib disposed on the inner surface of the connecting case. The connecting case includes a lateral opening configured to allow a user to remove a ring by pushing the ring laterally out of the connecting case. In this configuration, each of the plurality of rings is easily removable from and replaceable within the connecting case.

### BRIEF DESCRIPTION OF THE DRAWINGS

The accompanying drawings, which are incorporated in and constitute a part of the specification, illustrate the presently preferred embodiments and methods of the invention, and together with the general description given above and the detailed description of the preferred embodiments given below, serve to explain the principles of the invention.

FIG. 1 is a side elevation view of an exemplary embodiment of a stackable ring according to the present invention.

FIG. 2 is atop plan view the stackable ring of FIG. 1.

FIG. 3A is a side elevation view of an exemplary embodiment of a connecting case for holding the stackable rings of FIG. 1 according to the present invention.

FIG. 3B is a top plan view of the connecting case of FIG. 3A.

FIG. 4 is a front elevation view of an embodiment of the assembled collection apparatus of the present invention, showing the connecting case of FIG. 3A holding three of the stackable rings of FIG. 1.

FIG. 5A is a side elevation view of the assembled collection apparatus of FIG. 4.

FIG. 5B is a top plan view the assembled collection apparatus of of FIG. 5A.

FIG. 6 is a front elevation view of an embodiment of the assembled collection apparatus of the present invention, showing the connecting case of FIG. 3A holding five of the stackable rings of FIG. 1.

FIG. 7 is a side elevation view of the assembled collection apparatus of FIG. 6.

FIG. 8 is an exploded view of the stackable ring of FIG. 1.

FIG. 9 is an exploded perspective view of the collection apparatus 6.

FIG. 10 is a perspective view of the assembled collection apparatus of FIG. 6.

FIGS. 11A-F show cross-sectional side views of various inner sleeve configuration options for the stackable rings in accordance with the present invention.

### DESCRIPTION

Reference in this application is made to presently preferred embodiments of the invention. While the invention is described more fully with reference to these examples, the invention in its broader aspects is not limited to the specific details, representative devices, and illustrative examples shown and described. Rather, the description is to be understood as a broad, teaching disclosure directed to persons of ordinary skill in the appropriate arts, and not as limiting upon the invention.

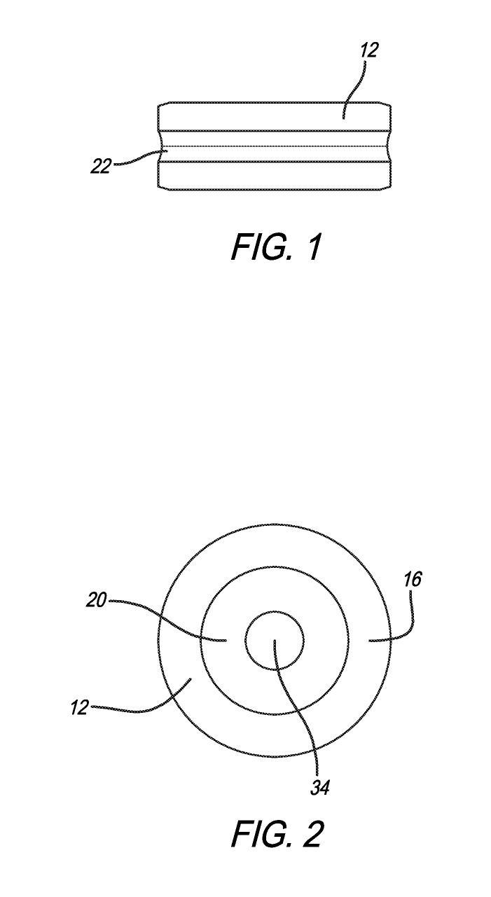

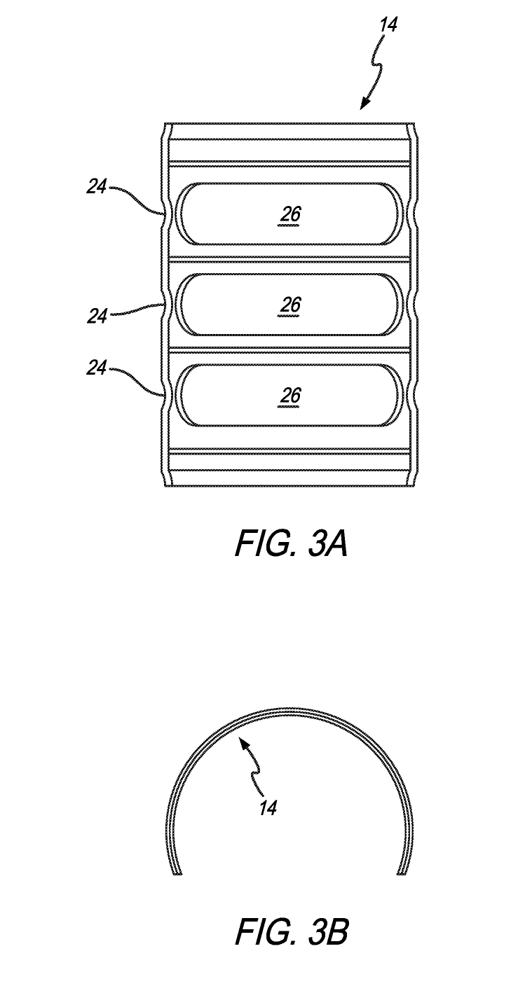

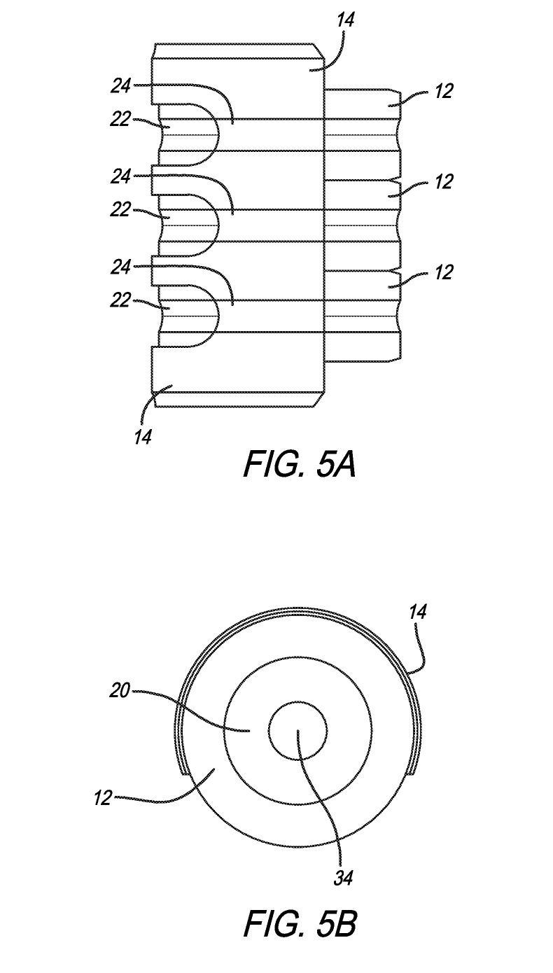

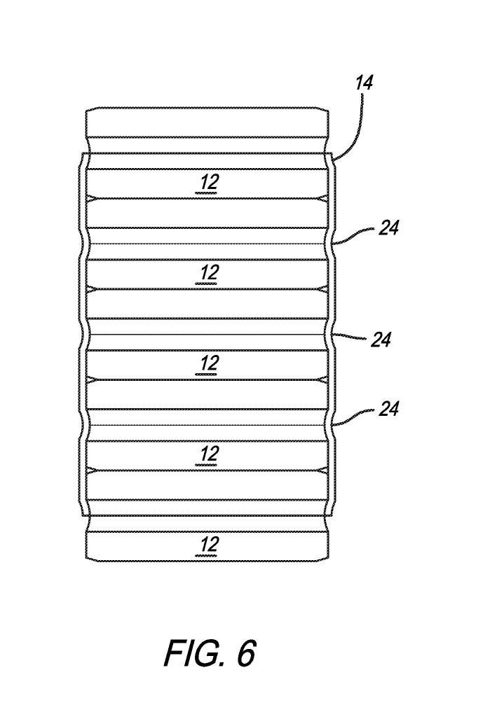

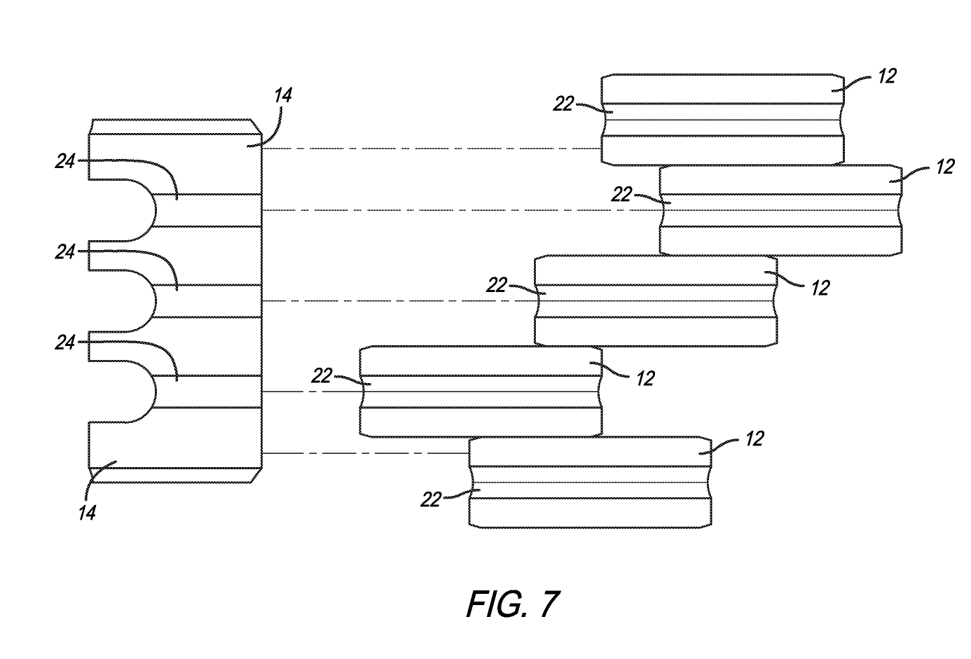

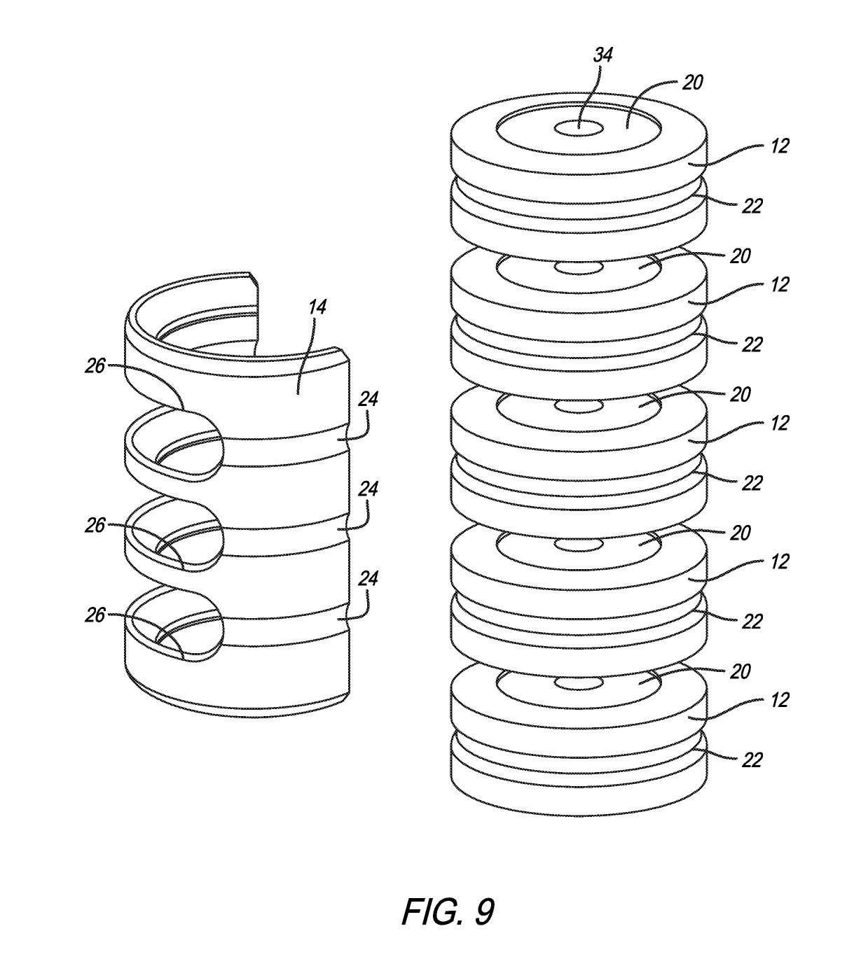

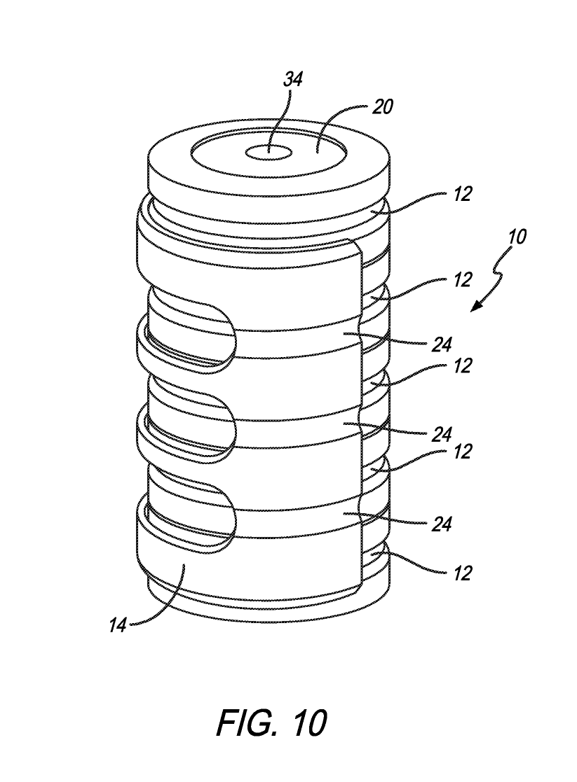

Referring generally to FIGS. 1-10, a presently preferred embodiment of a sperm collection apparatus 10 according to the invention includes a plurality of cups or rings 12 that are held in a concentric stacked relationship by a connecting case 14. The connecting case 14 can be made of a hard plastic that is sufficiently flexible to allow it to clip onto and hold the rings 12. In the embodiment shown, the connecting case 14 extends around the circumference of each of the plurality of rings by more than 180 degrees (see, e.g. FIG. 3B and FIG. 5B). The connecting case 12 includes a set of inwardly projecting ribs 24, each of which is configured to mate with a peripheral groove 22 of a ring 12 to restrict longitudinal movement of the ring 12 when it is inserted into the connecting case 14. The connecting case 14 also includes a plurality of openings 26 to allow a user to remove a ring 12 by pushing it laterally out of the case 14. In this configuration, each of the rings 12 is easily removable from and replaceable within the connecting case 14.

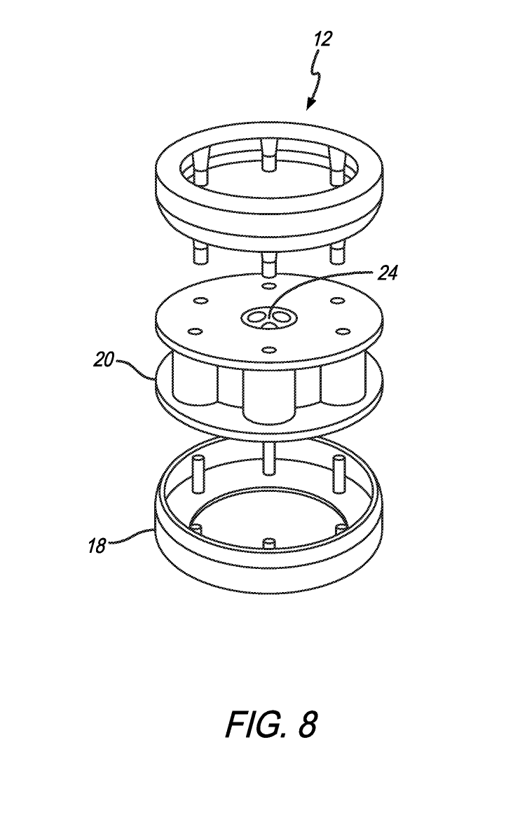

As shown in FIG. 8, each ring 12 has an inner sleeve 20 that is held within a ring top portion 16 and a ring bottom portion 18. When assembled, each ring 12 includes the peripheral groove 22.

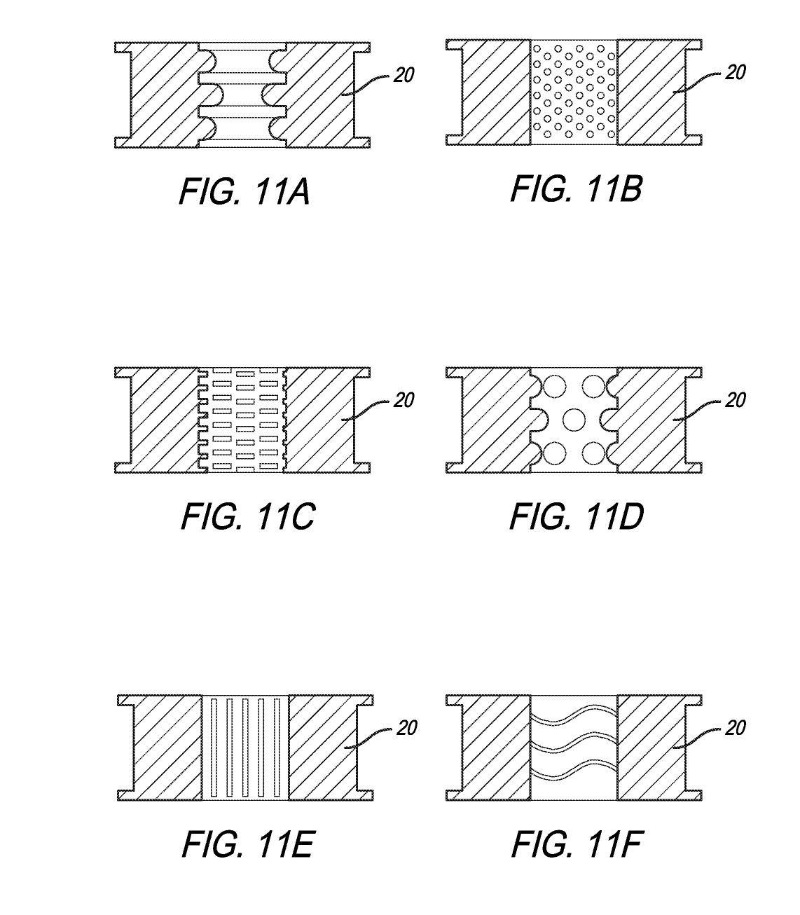

The inner sleeve 20 is made from a gel-like material, as is known in the art, and defines an internal insertion channel 34 that is sized for closely receiving a user's penis. The insertion channel 34 can be configured with varying diameters, geometries, projections, ridges and folds along its length to provide a user with the sensation of a life-like canal and with enhanced sensations during use. Various configuration options for inner sleeves 20 can be seen in FIG. 11.

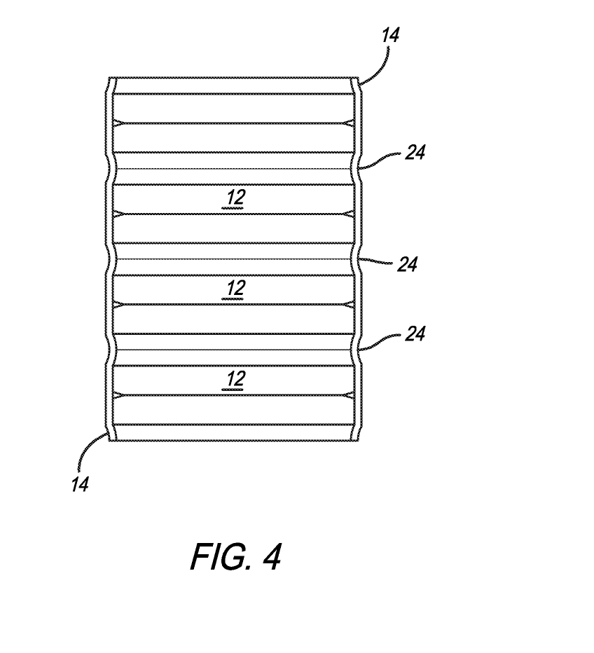

As can be seen in FIGS. 4-7 and 10, the connecting case 14 is configured to hold a plurality of the stackable rings 12 in a concentric stacked relationship. By assembling a plurality of rings 12 into such a stacked relationship, the concentric insertion channels 34 define an internal elongated insertion channel that is sized for closely receiving a user's penis. By using a varying number of rings 12, the length of this elongated channel can be varied. Also, by using rings 12 having different inner sleeve configurations, the channel can be configured with varying diameters, geometries, projections, ridges and folds along its length to provide a user with different sensations during use.

Having read this disclosure, it will also be understood by those having skill in the art that modifications may be made to the invention without departing from its spirit and scope. Therefore, the invention in its broader aspects is not limited to the specific details, representative devices, and illustrative examples shown and described. Accordingly, departures may be made from such details without departing from the spirit or scope of the general inventive concept.

What is claimed is:

1. A sperm collecting apparatus comprising:
a plurality of rings wherein each ring includes an inner sleeve comprising a gel-like material and wherein the inner sleeve defines an internal insertion channel portion that is sized for closely receiving a human penis;
wherein each ring of the plurality of rings is configured so that it can be placed in a concentric stacked relationship with each of the other of the plurality of rings so that the insertion channel portions of each of the rings form an elongated insertion channel; and
a connecting case configured to hold the plurality of rings in the concentric stacked relationship;
whereby the length of the insertion channel can be adjusted by varying the number of rings in the concentric stacked relationship.

2. The collecting apparatus of claim 1 wherein the connecting case extends around the circumference of each of the plurality or rings by 180 degrees or more.

3. The collecting apparatus of claim 1 wherein the connecting case comprises a hard plastic that is sufficiently flexible to allow the connecting case to clip onto and hold each of the plurality of rings.

4. The collecting apparatus of claim 1 wherein the inner sleeve of each ring is held within a ring top portion and a ring bottom portion.

5. The collecting apparatus of claim 1 wherein each of the plurality of rings is removable from and replaceable within the connecting case.

6. The collecting apparatus of claim 1 wherein each of the plurality of rings includes a peripheral groove configured to mate with a rib disposed on an inner surface of the connecting case.

7. The collecting apparatus of claim 1 wherein the connecting case comprises of an opening configured to allow a user to remove a ring from the connecting case by pushing the ring laterally out of the connecting case.

8. A sperm collecting apparatus comprising:
a ring including an inner sleeve comprising a gel-like material, wherein the inner sleeve defines an internal insertion channel portion that is sized for closely receiving a human penis;
wherein the ring is configured so that it can be disposed in a concentric stacked relationship with one or more other similar rings so that the insertion channel portions of the stacked rings form an elongated insertion channel; and
wherein the ring is configured to be held in the concentric stacked relationship by a connecting case;
whereby the length of the insertion channel can be adjusted by varying the number of rings in the concentric stacked relationship.

9. The sperm collecting apparatus of claim 8 wherein the inner sleeve of the ring is held within a ring top portion and a ring bottom portion.

10. The sperm collecting apparatus of claim 8 wherein the ring is configured to be removable from and replaceable within the connecting case.

11. The sperm collecting apparatus of claim 8 wherein the ring includes a peripheral groove configured to mate with a rib disposed on an inner surface of the connecting case.

12. A sperm collecting apparatus comprising:
a connecting case configured to hold a plurality of rings in a concentric stacked relationship;
wherein each ring of the plurality of rings includes an inner sleeve comprising a gel-like material and wherein the inner sleeve defines an internal insertion channel portion that is sized for closely receiving a human penis; and
wherein the connecting case is configured to hold the plurality of rings in the concentric stacked relationship so that the insertion channel portions of each of the rings form an elongated insertion channel;
whereby the length of the insertion channel can be adjusted by varying the number of rings in the concentric stacked relationship.

13. The sperm collecting apparatus of claim 12 wherein the connecting case is configured to extend around the circumference of each of the plurality of rings by 180 degrees or more.

14. The sperm collecting apparatus of claim 12 wherein the connecting case comprises a hard plastic that is sufficiently flexible to allow the connecting case to clip onto and hold each of the plurality of rings.

15. The sperm collecting apparatus of claim 12 wherein the connecting case comprises a plurality of inwardly projecting ribs, each of which is configured to mate with a peripheral groove in one of the plurality of rings to restrict longitudinal movement of the ring when the ring is inserted into the connecting case.

16. The sperm collecting apparatus of claim 12 wherein the connecting case comprises a plurality of openings, each of which is configured to allow a user to remove one of the plurality of rings by pushing the ring laterally out of the connecting case.
